(19) United States
(12) Patent Application Publication
(10) Pub. No.: US 2006/0280854 A1
(43) Pub. Date: Dec. 14, 2006

(54) Maillard reaction compounds of cysteine and a sugar having meatlike flavour

(76) Inventors: Kris De Roos, Hoevelaken (NL); Kees Wolswinkel, Scherpenzeel (NL)

Correspondence Address: CURATOLO SIDOTI CO., LPA, 24500 CENTER RIDGE ROAD, SUITE 280, CLEVELAND, OH, 44145, US

(21) Appl. No.: 10/567,723

(22) PCT Filed: Aug. 13, 2004
(86) PCT No.: PCT/CH04/00504

§ 371 Date: Jul. 19, 2006

(30) Foreign Application Priority Data
Aug. 20, 2003 (GB) 03194636

(51) Int. Cl. A23L 1/40; C07C 321/02

(52) U.S. Cl. 426/589; 562/557

(57) ABSTRACT

The Amadori compounds of cysteine of Formula I 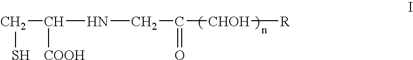 in which R is hydrogen when n=1-4, or methyl when n=0-3. The compounds are useful for conferring meaty tastes and aromas on foodstuffs.

This invention relates to a method of providing meat flavouring to foodstuffs and to novel compounds for providing such flavouring.

It is known that the formation of meat flavour proceeds via a sequence of steps known as the Maillard reaction. The sequence may be represented as follows:

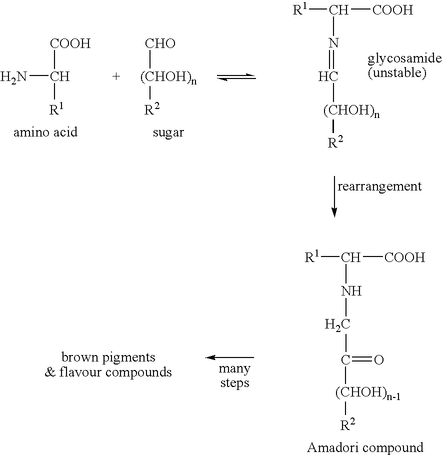

The Maillard reaction is also known as non-enzymatic browning. The Amadori compounds are key intermediates in the formation of flavours.

One amino acid for which no Amadori compound has hitherto been identified is cysteine (CH₂(SH).CH(NH₂).COOH). Cysteine plays an important role in the development of flavour in meat, and this is believed to result from its ability to release hydrogen sulphide under the conditions of the Maillard reaction (see, for example, Kobayashi and Fujimaki in Agric. Biol. Chem., 29, 698 81965). Numerous references have attributed to sulphur compounds a central role in the formation of meat flavours. However, unlike all other amino acids, cysteine has hitherto been believed to inhibit the Maillard reaction, because it tends to form relatively stable 2-glycosylthiazolidine-4-carboxylic acids, and this reaction competes with, and may even prevent, the formation of Amadori compounds.

It has now been found, surprisingly, that, not only is it possible to make Amadori compounds from cysteine, but also that these compounds are particularly effective at conferring meat flavour in foodstuffs. The invention therefore provides a compound of the formula I: 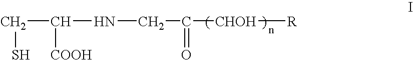
in which R is hydrogen when n=1-4, or methyl when n=0-3.

Although the compound is depicted as being of formula I, this form of the compound is in fact in equilibrium with two other forms, Ia and Ib, as shown below:

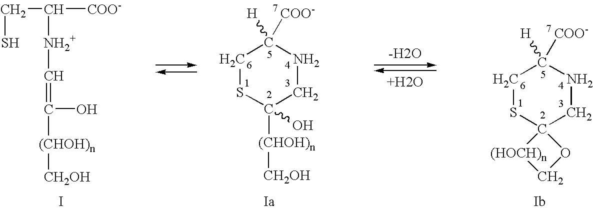

All three forms are included within the scope of this invention. Also included are all other isomeric forms, of which a number are possible (for example, when L-cysteine reacts with a D- or L-sugar).

The compounds may be prepared by known methods. Amino acids or their salts may be reacted with an aldose either in the presence of about 20% of water (see E. F. L. J. Anet, Austr. J. Chem. 10, 193-197 (1957)) or in an organic solvent (see K. Heyns and H. Paulen, Liebigs Ann. Chem., 622, 160 (1959)). In all preparations of Amadori compounds so far known, a reaction medium with low water content is essential to bring about the required condensation with aldoses to glycosyl amines. However, the preparation of the Amadori compounds of cysteine can also conveniently be carried out in dilute aqueous solutions or in mixtures of water with water-miscible solvents. It is not necessary to isolate the initially-formed 2-glycosylthiazolidine-4-carboxylic acids. The formation of the Amadori compounds from cysteine plus sugar or from 2-glycosylthiazolidine4-carboxylic acid is subject to general acid catalysis. For this reason, the reactions are preferably carried out in the presence of buffers having a pH of between 1 and 9, preferably between 2 and 6.

Although the Amadori rearrangement proceeds at room temperature, it is preferred to accelerate it by heating. The preferred temperature will depend on the solvents used and on the reactant concentrations, but the temperature generally lies between 60° and 110° C. At 90°-100° C., the preferred reaction time of the aqueous buffered reaction mixture is from 1-12 hours.

The sugars to be used for the present invention are aldoses with 3-6 carbon atoms, including aldopentoses, aldohexoses and deoxyaldohexoses. Specific sugar examples are the aldopentoses ribose, arabinose, xylose and lyxose, the aldohexoses glucose, mannose and galactose and the 6-deoxyaldohexoses rhamnose, fucose and chinovose. Mixtures thereof can also be used. In addition, all stereoisomeric forms of these sugars may be used.

The cysteine may be used as the compound itself or in the form of a derivative or a salt, which form free cysteine under the reaction conditions. Both L- and D-cysteine and mixtures thereof may be used. The molar proportions of the cysteine and the aldose may vary between 2:1 and 1:5, preferably between 1.2:1 and 1:1.5.

The buffers used may comprise mixtures of weak acids and bases or compounds that possess both an acidic and a basic function. Suitable acids include carboxylic acids and phosphoric acid. Typical examples of bases include the salts of the previously-mentioned acids, for example, their alkali metal salts of their salts with nitrogen-containing bases such as pyridine, α-picoline and tertiary amines. Primary and secondary amines may also be used, but preferably only if the reaction medium contains a large excess of water, to prevent the competitive reactions of these amines with the aldose to glycosylamines and Amadori rearrangement compounds of these amines. Examples of compounds with both acidic and basic functions are amino acids or their salts, for example glycine, proline and monosodium glutamate, or ribonucleotides or their salts, such as disodium inosine-5′-monophosphate. The buffers are preferably used in concentrations of from 0.1 to 10 times the molar concentration of cysteine.

The Amadori compounds thus prepared may be isolated as crude products and purified by known methods to give the compounds in colourless, crystalline form.

The Amadori compounds of this invention may be used to impart a meat-like taste to foodstuffs, both meat and non-meat, by incorporation of the compounds or compositions containing them into the foodstuff and subsequent heating. The preferred heating time depends on the water activity, the temperature used and the nature of the Amadori compound, but typically it is about the time needed to fry or cook meat.

The invention therefore also provides a method of conferring a meaty flavour or aroma on a foodstuff, comprising the addition to the foodstuff before heating of a compound as hereinabove described. The invention additionally provides the use of a compound as hereinabove described to confer on a foodstuff a meaty flavour or aroma.

The concentration required may vary over a wide range, depending on the desired taste and aroma effects, but as a general guide, they are employed in concentrations of from 5 to 5000 ppm by weight of foodstuff in consumable form. Good results are obtained by using from 200-2000 ppm.

The Amadori compounds of this invention may be used in combination with other known food additives, such as flavour compounds, aroma enhancers, and the like.

The invention is further described with reference to the following non-limiting examples.

### EXAMPLE 1

### Preparation of Amadori Compound of L-cysteine and D-xylose

A solution of 60.5 g (0.50 mol) L-cysteine 82.5 g (0.55 mol) D-xylose and 50 g (about 0.60 mol) of a mixture of methylated pyridines in 750 ml demineralised water was heated to about 95° C. on a water bath in a 1 L round-bottomed flask equipped with a reflux condenser. After 0.5 h, 120 g (2.0 mol) acetic acid was added and the solution was heated for a further 6 h at 90° C. The conversion to Amadori compound was then about 25%. The solution was cooled to room temperature and the solution was then continuously extracted with ether to remove the methylated pyridines. The solution was passed through a 40×4 cm column of Bio-Rad AG 50W-X4 (H⁺) cation exchange resin. The column was washed with 1 L water and the absorbed substances were displaced from the column with 0.3N ammonia. Fractions containing more than 50% of the compound were combined and evaporated in vacuo to small volume. After the addition of just enough ethanol to cause no cloudiness, crystalline compound separated. The pure compound did not melt but decomposed above 170° C.

Found: C 37.87; H 5.96; N 5.51; S 12.65; O 37.80.

Calc. for C₈H₁₅NSO₆ (253.27): C 37.94; H 5.97; N 5.53; S 12.66; O 37.90.

The product was dried prior to element analysis (in vacuo over P₂O₅, 18 h at 69° C.).

The results of a ¹³C NMR spectrum (in D₂O; δ in ppm, referenced to sodium 3-trimethylsilyl [2,2,3,3-²H₄]-propionate) were as follows:

| Major isomer | Minor isomer | | assignment |
|---|---|---|---|
| 26.9(t) | 25.6(t) | | C6 |
| 53.8(t) | 50.2(t) | | C3 |
| 61.5(d) | 57.0(d) | | C5 |
| 65.2(t) | 65.2(t) | | CH₂OH |
| 71.9(d) | 71.6(d) | | |
| | | {close oversize brace} | CHOH |
| 75.0(d) | 76.6(d) | | |
| 81.5(s) | 80.9(s) | | C2 |
| 173.4(s) | 173.0(s) | | carboxyl carbon |

### EXAMPLE 2

### Preparation of Amadori Compound of L-cysteine and D-xylose

L-cysteine.HCl monohydrate (17.6 g; 0.1 mol) was dissolved in 50 ml of demineralized water contained in a 150-ml round-bottomed flask. Disodium hydrogen phosphate (14.2 g; 0.10 mol) or 90% lactic acid (10 g; 0.10 mol) was added and the pH of the resulting solution adjusted to 5.0 by slowly adding with stirring an aqueous 50% sodium hydroxide solution. The sugar (0.11 mol) was then added and the mixture stirred for 1 h at 50° C. to obtain an equilibrium mixture of thiazolidine, cysteine and sugar. After cooling to room temperature, the pH was re-adjusted to 5.4 by addition of 50% sodium hydroxide solution. The water content was adjusted by evaporation in vacuo to 30%. The flask was then equipped, via a double neck adapter, with a mechanical stirrer and a reflux condenser, and heated with stirring for 7 h in a thermostated water bath of 80° C. The conversion to Amadori compound is 35%. Isolation was performed as described in Example 1.

### EXAMPLE 3

### Continuous Process for Production of Amadori Compound of L-cysteine and D-xylose

The continuous reactor consisted of a 45×2.5 ID cm glass column with temperature control jacket packed with Bio-Rex 9 anion exchange resin (OH⁻), 100-200 mesh. A restriction consisting of a 500×0.3 mm ID Teflon™ tubing was connected to the column outlet to prevent the formation of gas bubbles in the column. The column was thermostated at 90° C. A 50% thiazolidine solution, prepared by heating equimolar amounts of cysteine and xylose in water for 1 h at 50° C., was pumped through the column at a rate of 0.5 ml/min. Analysis of samples taken at regular time intervals showed that the conversion to Amadori compound increased from 12% after 2 hrs to 35% after 6 hrs. The yield of the corresponding batch process was 22.5%. The Amadori compound was isolated from the column eluate by evaporation in vacuo and crystallization of the residue from water-ethanol as in Example 1.

### EXAMPLE 4

### Preparation of the Amadori Compound of L-cysteine and D-ribose

The same equipment was used as was used in Example 1. 12.1 g (0.1 mol) of L-cysteine, 15 g (0.1 mol) of D-ribose and 27.2 g (0.2 mol) of potassium dihydrogen phosphate in 150 ml demineralised water were heated for 6 h at ca. 90° C. under nitrogen. The resulting brown solution was passed through a 35×2.5 cm column of Dowex™ 50W-X4 (H⁺) cation exchange resin. The neutral components were washed from the column with 1 L water and the nitrogen-containing substances were displaced from the column with 0.3N ammonia. Fractions containing more than 50% of the compound were combined and evaporated in vacuo to small volume. After the addition of just enough ethanol to cause no cloudiness, crystalline compound separated. The pure compound did not have a sharp melting point but decomposed at 132°-135° C. The results of a ¹³C NMR spectrum (in D₂O; δ a in ppm) were as follows:

| Major isomer | Minor isomer | | assignment |
|---|---|---|---|
| 26.8(t) | 25.7(t) | | C6 |
| 55.2(t) | 51.1(t) | | C3 |
| 61.9(d) | 57.3(d) | | C5 |
| 65.6(t) | 65.3(t) | | CH₂OH |
| 74.0(d) | 73.4(d) | | |
| | | {close oversize brace} | CHOH |
| 75.9(d) | 77.8(d) | | |
| 82.7(s) | 81.1(s) | | C2 |
| 174.0(s) | 173.5(s) | | carboxyl carbon |

### EXAMPLE 5

### Preparation of the Amadori Compound of L-cysteine and L-arabinose

The process of Example 3 was repeated with L-arabinose instead of D-xylose. The resulting crystalline material melted with decomposition at 171-173° C.

The results of a ¹³C NMR spectrum (in D₂O; δ in ppm) were as follows:

| Major isomer | Minor isomer | | assignment |
|---|---|---|---|
| 26.9(t) | 25.8(t) | | C6 |
| 54.9(t) | 51.7(t) | | C3 |
| 62.1(d) | 57.2(d) | | C5 |
| 65.4(t) | 65.6(t) | | CH₂OH |
| 73.7(d) | 73.9(d) | | |
| | | {close oversize brace} | CHOH |
| 77.7(d) | 76.2(d) | | |
| 81.1(s) | 82.8(s) | | C2 |
| 174.0(s) | 173.6(s) | | carboxyl group |

### EXAMPLE 6

### Preparation of the Amadori Compound of L-cysteine and D-glucose

An aqueous solution of 12.1 g (0.1 mol) of L-cysteine, 19.8 g (0.1 mol) of D-glucose and 11.5 g (0.1 mol) of L-proline was concentrated in vacuo to a syrup containing approximately 20% of water. The syrupy mixture was heated on a water bath at 95° C. for 2.5 h. HPLC analysis showed that, in addition to the Amadori compound of L-cysteine, the Amadori compound of L-proline was also formed, in the ratio of 3:1. The brown reaction mixture was dissolved in 500ml water and the Amadori compounds recovered as described in Example 2. The results of a ¹³C NMR spectrum (in D₂O; δ in ppm) were as follows:

| Major isomer | Minor isomer | | assignment |
|---|---|---|---|
| 27.1(t) | 25.9(t) | | C6 |
| 54.1(t) | 50.3(t) | | C3 |
| 61.6(d) | 57.2(d) | | C5 |
| 65.1(t) | 65.1(t) | | C6′ |
| 63.3(t) | 63.0(t) | | CH₂OH |
| 71.6(d) | 71.3(d) | | |
| 73.3(d) | 73.5(d) | {close oversize brace} | CHOH |
| 74.4(d) | 76.1(d) | | |
| 81.8(s) | 81.1(s) | | C2 |
| 173.6(s) | 173.0(s) | | carboxyl carbon |

### EXAMPLE 7

### Preparation of the Amadori Compound of L-cysteine and L-rhamnose

A solution of 3.3 g (0.30 mol) L-cysteine 49 g (0.27 mol) L-rhamnose monohydrate and 30 g (about 0.0 mol) of a mixture of methylated pyridines and 72 g (1.20 mol) acetic acid in 450 ml demineralised water was heated for 8 h on a steam bath in a 1 L round-bottomed flask equipped with a reflux condenser. The resulting brown reaction mixture was purified as described in Example 1. The resulting crystalline compound decomposed at 183° C. The results of a ¹³C NMR spectrum (in D₂O; δ in ppm) were as follows:

| Major isomer | Minor isomer | assignment |
|---|---|---|
| 21.1 or 21.2(q) | 21.1 or 21.2(q) | CH₃ |
| 26′9(t) | 25.9(t) | C6 |
| 54.3(t) | 50.8(t) | C3 |
| 62.1(d) | 57.0(d) | C5 |
| 69.9(d) | 69.7(d) | |
| 75.2(d) | 74.7(d) | CHOH |
| 75.8(d) | 75.4(d) | |
| 81.3(s) | 81.8(s) | C2 |
| 173.9(s) | 173.5(s) | carboxyl carbon |

### EXAMPLE 8

### Sensory Evaluation of Amadori Compounds

A beef broth was prepared. Four portions of this broth were treated as follows:
Portion A: no treatment (control);
Portion B: addition of 480 ppm (4.0 mmol/L) L-cysteine+552 ppm (4.0 mmol/L) D-xylose;
Portion C: addition of 1000 ppm (4.0 mmol/L) 2-xylosylthiazolidine-4-carboxylic acid (prepared according to R Bognár et al, Liebigs Ann. Chem., 738, 68 (1970);
Portion D: addition of 1000 ppm (4.0 mmol/L) of the Amadori compound of Example 1.

The mixtures were bottled and sterilised in an autoclave for 1 h at 100° C. On sensory evaluation, Mixture D was preferred, because of its much stronger meaty taste. Moreover, it lacked the cysteine-like off-taste of Mixture B and the hydrogen sulphide-like off-odour of Mixture C.

### EXAMPLE 9

### Test in a Gravy

A gravy was prepared by heating 100 g of commercial, flavoured margarine to 160° C. On reaching this temperature, 35 ml water was added. This was used as an unflavoured control.

A second gravy was prepared by adding 100 mg (1000 ppm) of the Amadori compound of Example 2 to 100 g of margarine and heating to 160° C. When this temperature was reached, 35 ml water was added.

A taste comparison revealed a considerable preferment for the gravy containing the Amadori compound, because of its pronounced, nicely roast, meaty taste.

1. A compound of the formula I: 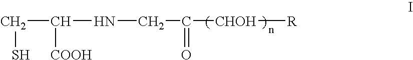
in which R is hydrogen when n=1-4, or methyl when n=0-3.

2. A method of preparing a compound according to claim 1, comprising the reaction of cysteine with a sugar in either aqueous solution or in a mixture of water and water-miscible solvents.

3. A method according to claim 2, in which the reaction is accelerated by heating.

4. A method according to claim 2, in which the reaction is carried out in the presence of a buffer having a pH of from 1-9.

5. A method of conferring a meaty flavour or aroma on a foodstuff, comprising the addition to the foodstuff before heating of a compound according to claim 1.

6. Use of a compound according to claim 1 to confer on a foodstuff a meaty flavour or aroma.

7. A method according to claim 3, in which the reaction is accelerated by heating to a temperature between 60° and 100° C.

8. A method according to claim 4, in which the buffer has a pH of from 2-6.

9. A method according to claim 5, wherein the compound is added in a concentration ranging from 5-5000 ppm by weight of foodstuff in consumable form.

10. A method according to claim 5, wherein the compound is added in a concentration ranging from 200-2000 ppm by weight of foodstuff in consumable form.
